(19) United States
(12) Patent Application Publication
(10) Pub. No.: US 2020/0197914 A1
(43) Pub. Date: Jun. 25, 2020

(54) PREPARATION OF METAL-IN-HOLLOW-ZEOLITE-BASED CATALYST FOR SELECTIVE BENZENE ALKYLATION

(71) Applicant: SABIC Global Technologies B.V., Bergen op Zoom (NL)

(72) Inventors: Ugo RAVON, Thuwal (SA); Abdulrahman M. ALHAZMI, Thuwal (SA); Abdullah ALTHOBAITY, Thuwal (SA); Omar A. ABED, Thuwal (SA); Khalid ALBAHILY, Thuwal (SA)

(21) Appl. No.: 16/614,419

(22) PCT Filed: May 10, 2018
(86) PCT No.: PCT/IB2018/053236

§ 371 Date: Nov. 18, 2019

Related U.S. Application Data
(60) Provisional application No. 62/507,919, filed on May 18, 2017.

(51) Int. Cl. B01J 29/035; B01J 37/02; B01J 37/06; B01J 37/08; C07C 2/70; B01J 35/00; B01J 35/02; B01J 35/08; B01J 29/46

(52) U.S. Cl. CPC B01J 29/0356; B01J 37/0201; B01J 37/06; B01J 37/0236; B01J 37/088; C07C 2/70; B01J 2229/20; B01J 35/026; B01J 35/08; B01J 29/46; C07C 2521/08; C07C 2521/04; B01J 35/0006

(57) ABSTRACT

The invention is directed to hollow zeolite encapsulated metal particle catalysts where the metal particle is contained in the hollow of the zeolite, their preparation method by depositing metal particle precursors and subsequent removal of said metal particle precursors from the surface of the hollow zeolite while retaining those in the cavity of the hollow zeolite, and the catalysts' use in selective benzene alkylation.

### CROSS-REFERENCE TO RELATED APPLICATIONS

This application claims the benefit of priority of U.S. Provisional Patent Application No. 62/507,919 filed May 18, 2017, which is hereby incorporated by reference in its entirety.

### BACKGROUND

In view of the current environmental challenges there is an urgent need to develop a more sustainable chemical industry through more efficient chemical transformations and processes. Improvements in these chemical transformations and processes can include the (i) enhancement of the reaction yield and/or selectivity, (ii) reduction of operating cost, and (iii) use of more suitable reactants and catalysts. One approach to addressing the inefficiencies and cost of current processes is by developing new highly selective and cost-effective catalysts, as well as more efficient and cost effective methods of making the catalyst.

Zeolites are a family of crystalline materials that can be used in the design and development of new catalysts and catalyst supports. Zeolites have a porous structure that can accommodate a wide variety of cations, such as Na⁺, K⁺, Ca²⁺, Mg²⁺ and others. Currently, there are more than 200 different zeolites types registered and referenced by the International Zeolite Association (IZA). Due to their porosity and their high surface area, zeolites are used as catalyst and/or a catalyst supports. Metals can be deposited in the pores and on the surface of a zeolite, or incorporated into the zeolite framework in order to enhance specific reactions. A number of processes have been described for metallic particle dispersion on the zeolite surface; however, the metallic particles can diffuse through the pores rendering the catalyst unstable. In most of the cases, this leaching effect is the main deactivation process.

Encapsulation of metal nanoparticles in a zeolite structure can improve the physical and catalytic properties of the zeolite. Encapsulation can protect the individual nanoparticles from contact with other nanoparticles, thereby preventing sintering of the nanoparticles when subjected to elevated temperatures. Post-treatment deposition of nanoparticles inside zeolites has been reported, but the post-synthesis treatments result in nanoparticles in the cages and/or in the pores of the zeolite. It can be difficult to control the size, location, and retention of the nanoparticles in these post-treatment zeolite compositions. General methods for the stabilization of metal nanoparticles are far from being fully developed, although for some specific systems it has been achieved by optimizing the interaction of nanoparticles with a support material or by encapsulation of the metal particles. However, these known catalytic systems are in general very expensive and difficult to synthesize and they cannot be produced on an industrial scale.

Zeolite catalyst have been developed for use in alkylation of benzene in processes for producing ethylbenzene and cumene. Acid catalysts of both the zeolitic and non-zeolitic type are negatively influenced by the presence of water, which is produced when ethanol is used as alkylating agent for benzene. Thus, the use of ethanol and an acid catalyst for the alkylation of benzene to form ethylbenzene has proven to be non-practicable from an industrial point of view due to the negative effects of water on catalyst performance.

Other zeolites have been described for use in benzene alkylation reactions. For instance, Johney et al., (Indian Journal of Technology, 1977, 15:486-89) describe the alkylation of benzene with ethanol at atmospheric pressure in the presence of variably substituted 13-X zeolites; however, the activity of these catalysts is not very high, and rapidly decreases. Chandawar et al. (Applied Catalysis, 1982, 4:287-95) describe the alkylation of benzene with ethanol in the presence of ZSM-5 zeolite; however, acceptable conversions are only obtained at extremely high temperatures. In another instance, Corma et al., (Journal of Catalysis, 2002, 207:46-56) describe the alkylation of benzene with ethanol, in the presence of ITQ-7 zeolite and beta zeolite; however, the reaction leads to the formation of poly-alkylated aromatic compounds, in addition to xylene and other undesired products.

Thus, there is still a need for new highly selective and cost-effective catalysts, as well as new more efficient methods for making such catalysts. In particular, there is a need for improved catalysts for use in the efficient and selective production of alkylated benzenes.

### SUMMARY

A solution to the some of the problems discussed above concerning non-optimum zeolite catalyst has been discovered. The solution is premised on zeolite encapsulated particle compositions produced by these methods and methods of making such a zeolite. The catalyst preparation methods described herein provide for a catalyst comprising a hollow zeolite with an encapsulated metal particle, where the metal particle is contained in the core of the zeolite that is within the inner surface of the hollow zeolite. This catalyst addresses many of the outstanding problems associated with zeolite catalysts and provides improved selectivity and stability in a multitude of reactions, particularly alkylation of benzene.

Certain embodiments are directed to a catalyst comprising a metal particle encapsulated within a hollow zeolite. Metal can be located on the surface of the zeolite, in the pores of the zeolite, incorporated into the zeolite framework, or encapsulated in the hollow/core of the zeolite structure. In certain aspects, the zeolite catalyst of the present invention can have less than 1.0, 0.5, 0.1, 0.01, 0.001, or 0.0001 wt. % metal (i) on the surface of the zeolite, (ii) in the pores of the zeolite, (iii) in the zeolite framework, or (iv) on the surface, in the pores, and in the zeolite framework. In certain aspects, the zeolite has no detectable metal on the surface, in the pores, and/or in the framework of the hollow zeolite. The metal particle can include one or more of copper (Cu), nickel (Ni), cobalt (Co), gold (Au), platinum (Pt), palladium (Pd), ruthenium (Ru), iron (Fe), titanium (Ti), iridium (Ir), or gallium (Ga), including aggregates, and/or alloys of one or more of these metals. In certain aspects, the metal particle can have a diameter of at least, at most, or about 1 to 30 nm. Certain embodiments are directed to a catalyst that includes a hollow zeolite encapsulated metal particle, where the metal particle is contained in the core of the hollow zeolite. The metal particle core can include at least 95, 96, 97, 98, 99, or 99.9 wt. % of the metal present in the hollow zeolite encapsulated metal particle. In particular aspects, the metal particle core includes at least 97 wt. % of the metal present in the hollow zeolite encapsulated metal particle.

Further aspects of the present invention are directed to methods for producing the hollow zeolite catalyst (e.g., a hollow zeolite encapsulated metal catalyst) of the present invention. A method can include: (a) depositing a metal particle precursor in a hollow zeolite material by contacting the hollow zeolite material with a metal particle precursor that permeates or is transported into the hollow zeolite material and deposits the metal particle precursor in the hollow zeolite forming a hollow zeolite encapsulated metal particle; (b) removing metal particle precursor from the surface of the hollow zeolite while retaining the deposited metal particle precursor in the cavity of the hollow zeolite by contacting the hollow zeolite with the encapsulated metal particle with a non-permeating wash solution forming a loaded hollow zeolite; (c) drying the loaded hollow zeolite; and (d) calcining the loaded hollow zeolite at a temperature of 450° C. to 650° C. to form a hollow zeolite catalyst that includes a metal particle containing core. In certain aspects, steps (a) and (b) are repeated 2, 3, 4, 5, 6, 7, 8, 9, 10 or more times prior to the calcination step. In certain aspects the zeolite framework contains a reduced concentration or metal (0.001, 0.05, 0.1, 0.5 1, 2, 5, to 10 wt. % or less, including all values and ranges there between) to no detectable metal in the zeolite framework or lattice surrounding or encapsulating the metal particle. The metal particle can include iron (Fe), silver (Ag), Au, Ti, Cu, zinc (Zn), Co, manganese (Mn), magnesium (Mg), Ni, Pt, Pd, Ir, Ru, Ga, aluminum (Al), tungsten (W), bismuth (Bi), vanadium (V), indium (In), or combinations or alloys thereof. In certain aspects, the metal particle precursor can be a metal oxide or metal salt. In a further aspect, the metal particle precursor can be a nitrate, chloride, sulfate, ammonium, acetate, or oxalate metal particle precursor. The metal particle precursor can be provided as a metal particle precursor solution. The metal particle precursor solution can include a solvent such as alcohol or water. In certain aspects, the solution is an ethanol or methanol solution of the metal particle precursor.

The depositing step can include wet impregnation, dry impregnation, vacuum impregnation, or ion exchange. In certain aspects, the depositing step can be a wet impregnation step. Non-limiting examples of the hollow zeolite can include MFI, *BEA, FAU, or MWW type zeolite. In certain aspects, the hollow zeolite to metal particle precursor weight ratio in step (a) is 4:1, 3:1, 2:1, to 1:2, 1:3, 1:4. In particular aspects, the hollow zeolite to metal particle precursor weight ratio in step (a) is 2:1 to 1:2. The method can further include forming a hollow zeolite by treating a zeolite with a corresponding template-hydroxide, which forms a first reaction mixture, and then heating the first reaction mixture to a temperature of 150° C. to 200° C. for 24 to 120 hours to form a hollow zeolite material. In certain aspects, the zeolite is a MFI type zeolite and the corresponding template-hydroxide is tetrapropylammonium hydroxide, a *BEA type zeolite and the corresponding template-hydroxide is tetraethylammonium hydroxide, a FAU type zeolite and the corresponding template-hydroxide is tetramethylammonium hydroxide, or a MWW type zeolite and the corresponding template-hydroxide is hexamethyleneimine hydroxide. In certain aspects, the catalyst is substantially aluminum free. In other aspects, the catalyst can have a silica to alumina weight ratio of about or at least 20:1 40:1, 50:1, 100:1, 200:1 500:1:1 to co. In a further aspect, the catalyst is substantially aluminum free, i.e., aluminum is at levels that are not detectable using standard x-ray diffraction techniques.

Other embodiments are directed to methods for alkylating benzene. A method can include contacting benzene with an alkylene (alkene) in the presence of a hollow zeolite catalyst, as described herein or as produced by the process described herein, at a temperature of at least, equal to, or between any two of 20, 40, 80, 100, 140, 180 to 200, 220, 240, 280, 300, and 350° C. In certain aspects, the alkene is ethylene and the product is ethylbenzene, or the alkene is propylene and the product is cumene.

The following includes definitions of various terms and phrases used throughout this specification.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The use of the term “or” in the claims is used to mean “and/or” unless explicitly indicated to refer to alternatives only or the alternatives are mutually exclusive, although the disclosure supports a definition that refers to only alternatives and “and/or.”

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The catalysts of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the catalysts of the present invention are their abilities to catalyze alkylation of benzene.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 is a representation of the different steps of the catalyst synthesis.

FIG. 2 shows an X-ray diffraction (XRD) pattern of an intact zeolite crystal structure of the present invention, * are attributed to Fe oxide using ICSD:16129, Hill et al., Chem. Mater., 2008, 20:4891 as a reference.

FIG. 3 is a N₂ isotherm at 77 K of hollow Silicalite-1 and 5.3% Fe hollow silicalite-1 of the present invention.

FIGS. 4A and 4B depict transmission electron microscopy (TEM) images for Fe hollow silicalite-1; (4A) Single hollow silicalite-1 encapsulating with iron oxide; (4B) 0.5 μm scale of Fe incorporated in hollow silicalite-1.

FIG. 5 depicts an energy-dispersive X-ray (EDX) analysis for the FIG. 4A TEM image.

FIG. 6 depicts a dark field microscopy analysis.

FIG. 7 is a comparison of the conversion and selectivity of ZSM5 Si/Al=23 catalyst and a hollow zeolite encapsulated metal particle catalyst in a benzene alkylation reaction. Illustrated is the GC/MS analysis of the recovered liquid demonstrating a selectivity for ethylbenzene.

### DESCRIPTION

Recently, a new hollow form of zeolite has been discovered. It is believed that this zeolite is obtained by a dissolution recrystallization process (Li et al., Chem. Commun., 2014, 50:1824). Typically, OH— of the TPA(OH) can migrate into the zeolite core and dissolve silica present in the core. Then, due to the presence of TPA+ located around the zeolite particle, the dissolved silica migrates from the zeolite particle and recrystallizes around this particle. So after several washing steps, a hollow zeolite is obtained. The common method (which is claimed to be the best) is to encapsulate metals particles inside the hollow concurrently with the formation of the hollow. The present invention provides a new method of encapsulating a metal particle in a hollow zeolite.

### I. HOLLOW ZEOLITE CATALYST FOR SELECTIVE ALKYLATION

Zeolites are the aluminosilicate members of the family of microporous solids known as “molecular sieves” mainly consisting of Si, Al, O, and metals including Ti, Sn, Zn, and so on. The term molecular sieve refers to a particular property of these materials, i.e., the ability to selectively sort molecules based primarily on a size exclusion process. This is due to a very regular pore structure of molecular dimensions. The maximum size of the molecular or ionic species that can enter the pores of a zeolite is controlled by the dimensions of the channels. These channels are defined by the ring size of the aperture, where, for example, the term “8-ring” refers to a closed loop that is built from eight tetrahedrally coordinated silicon (or aluminum) atoms and 8 oxygen atoms. These rings are not always perfectly symmetrical due to a variety of effects, including strain induced by the bonding between units that are needed to produce the overall structure, or coordination of some of the oxygen atoms of the rings to cations within the structure. Therefore, the pores in many zeolites are not cylindrical. The porous structure of zeolites can accommodate a wide variety of cations, such as Na⁺, K⁺, Ca²⁺, Mg²⁺ and others. These positive ions are rather loosely held and can readily be exchanged for others in a contact solution.

Some of the more common mineral zeolites are analcime, chabazite, clinoptilolite, heulandite, natrolite, phillipsite, and stilbite. An example of the mineral formula of a zeolite is: Na₂Al₂Si₃O₁₀×2H₂O, the formula for natrolite. Natural zeolites form where volcanic rocks and ash layers react with alkaline groundwater. Zeolites also crystallize in post-depositional environments over periods ranging from thousands to millions of years in shallow marine basins. Naturally occurring zeolites are rarely pure and are contaminated to varying degrees by other minerals, metals, quartz, or other zeolites. For this reason, naturally occurring zeolites are excluded from many important commercial applications where uniformity and purity are essential.

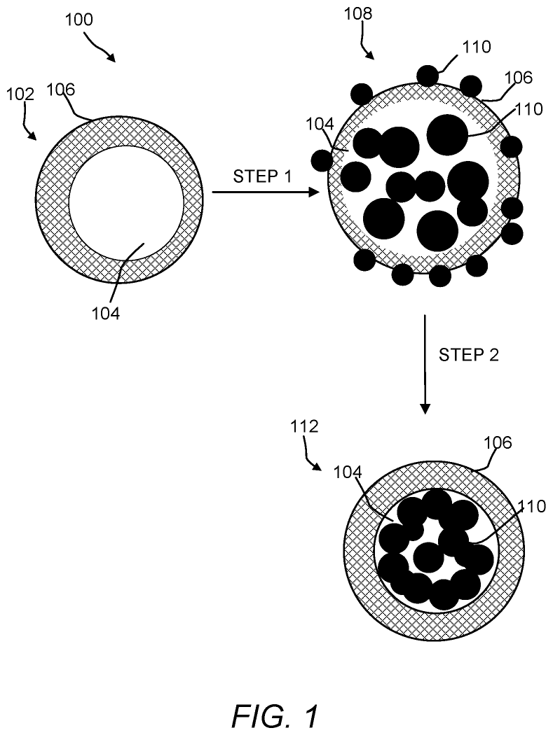

It has been discovered that specific metal oxide particles encapsulated within a hollow zeolite (e.g., a silicalite-1) can enhance the selectivity of chemical processes, which can be applied to particular reactions such as the alkylation of benzene. The main concern with encapsulation is metal incorporation or intercalation into the zeolite framework during the dissolution-recrystallization process. Indeed, during the hollow zeolite protocol of Tuel et al. the metal salts are impregnated/intercalated in silicalite-1. Then TPA(OH), tetra propyl ammonium hydroxide, is added to carry out the hollow zeolite synthesis. During this step metal complexes and dissolved silica are present in the mixture. Under this condition, metal can be incorporated into the framework and be a part of the zeolite crystal. As an example, the work of Occelli or Taramasso describe the incorporation of Ti into TS-1 structure or Ga into FAU structure zeolite respectively. To overcome the issue of the incorporation, a modification of the protocol was needed. The new protocol where the hollow zeolite formation is carried out before metal incorporation, see for example FIG. 1.

By following the procedure described herein, metal complexes and dissolved silica are not in contact with each other, which reduces or prevents metal incorporation into the zeolite structure. As a result, the zeolite framework will have less than 10, 5, 2, 1, 0.5, 0.1, 0.01, 0.001, or 0.0001 wt. % metal incorporated or intercalated in the zeolite framework. Referring to FIG. 1, in method 100, hollow zeolite 102 having hollow portion 104 and outer surface 106 can be contacted in step 1 with a metal precursor solution to form hollow zeolite 108 having metal precursor 110 localized within the hollow portion 104 and on external zeolite surface 106. In step 2, a washing step can be included to remove metal precursors 110 localized at the external surfaces. A washing step can be done with a solvent able to dissolve the metal complex, but not substantially enter or permeate inside the pore of the zeolite. Because zeolites such as silicalite-1 (type MFI zeolite) are hydrophobic, water, for example, can be used for the washing step. Water can dissolve the metal complexes without diffusing through the pore of the zeolite. In certain aspects, the hollow zeolite can be contacted 2, 3, 4, 5, 6, or more time with a metal precursor solution/wash cycle prior to catalyst calcination to produce hollow zeolite 112 only having metal precursors 110 in hollow portion 104. In some embodiments, hollow zeolite 112 can be calcined to convert the metal precursor to metal oxides. The hollow zeolite with an encapsulated metal particle can be calcined, for example, 4, 6, 8, 10, 12, or more hours at a temperature of at least, equal to, or between any two of 500, 520, 540, 560, and 580° C. The zeolite, but is not limited to, have a metal content of 1, 2, 3, 4, 5, 6, 7, 8, 9, 10 weight percent (wt. %) of the hollow zeolite encapsulated metal particle catalyst. In certain aspects, the catalyst can have a metal oxide:zeolite weight ratio of 0.01, 0.05, to 0.10. The metal can be loaded at 0.5 to 10 wt. %. In certain instances, the zeolite can have a reduced amount to no Al in the zeolite framework and still encapsulate an Al containing particle or core.

A. Hollow Zeolite

A hollow zeolite can be formed by treating a zeolite with the corresponding template in the hydroxide form, e.g., tetrapropylammonium hydroxide (TPA(OH), Aldrich) for a MFI zeolite structure. The zeolite mixture is heated to an appropriate temperature for an appropriate time forming a hollow zeolite. The hollow zeolite can then be recovered and washed to remove excess template. The washed hollow zeolite is dried and calcined in order to clean the zeolite pores. In certain aspects the hollow zeolite is a hollow MFI, *BEA, or FAU zeolite. In certain aspects the Si/Al ratio is 20 to co.

B. Metal Particles

Metal particles encapsulated in the zeolite can be formed from metal particle precursors. A metal particle precursor can include metal ions or a source of metal ions, such as a metal-containing salt (i.e., a metal precursor). Non-limiting examples of salts include a nitrate, a chloride, a sulfate, an ammonium, an acetate, and/or an oxalate. For example, in embodiments, the metal ion portion of the metal precursor can be Cu, Ni, Co, Au, Pt, Pd, Ru, Fe, Ti, Ir, Ga, as well as aggregates, alloys, or clusters of metals and any combination of these metals, such as Fe/Pt. In certain aspects, for example, the metal ions can be provided by dissolution of a metal salt in an appropriate solvent, e.g., a non-aqueous polar solvent. In certain aspects, the metal precursor can be dissolved in a water miscible solvent (e.g., alcohol such as ethanol, methanol, etc.) water, or mixtures thereof. In specific embodiments, the metal ions have a concentration in the solution from 10⁻³ M to 0.5 M and all ranges and values there between. The metal precursor(s) can be deposited in the zeolite hollow by wet impregnation, dry impregnation, vacuum impregnation, ion exchange, or other known methods. The metal particle can be formed in the zeolite hollow, and thus the enveloped metal particle, includes, but is not limited to, a particle comprising Cu, Ni, Co, Au, Pt, Pd, Ru, Fe, Ti, Ir, Ga, or any alloy thereof. In certain aspects the enveloped metal particle will have an average diameter of 1 to 30 nm, or at least, equal to, or between any two of 1, 5, 10, 15, 20, 25, and 30 nm.

C. Selective Alkylation of Benzene

Ethylbenzene is an important intermediate product of basic chemical industries. It is mainly used as precursor for the production of styrene, which in turn is useful as an intermediate in the preparation of styrene polymers and copolymers. The industrial synthesis of styrene can include the steps of alkylation of benzene to ethylbenzene and the transformation of ethylbenzene into styrene by a dehydrogenation reaction. In the petrochemical industry, slurry reactors with mainly *BEA zeolite catalyst are used for the alkylation of benzene with ethylene to produce ethylbenzene.

Cumene is an intermediate of the phenol synthesis, which is economically even more attractive than styrene. The process to produce cumene process is similar to the ethylbenzene process. Cumene can be synthesized by alkylation of propylene with benzene. Like the ethylbenzene (EB) process, the cumene process suffers due to the production of byproducts.

The hollow zeolite encapsulated metal catalyst can be used in the alkylation of benzene. The process of benzene alkylation can be carried out in the gas phase, or in liquid or mixed phase, and batch wise, or in semi-continuous or continuous mode. The reaction temperature can range from 10° C. to 400° C., and in certain aspects from 20° C. to 350° C. or any value or range there between. The process can be performed at a pressure from 1 MPa to 5 MPa (10 to 50 atm), in certain aspects from 2.5 MPa to 3.5 MPa (25 to 35 atm). The weighted hourly space velocity (WHSV) at which the reactants can be fed to the reaction can be from 0.1 to 200 hours⁻¹ and preferably of from 1 to 10 hours⁻¹. In certain aspects, a method for alkylating benzene can include contacting benzene with an alkene in the presence of a hollow zeolite catalyst of the present invention (i.e., a zeolite having an embedded metal particle) at a temperature of 20 to 350° C. and any value or range there between. In certain aspects, the molar ratio of benzene to alkene (benzene:alkene) in the feedstock for the reaction can be in the range of from 20, 15, 10, 9, 8, 7, 6, 5, 4, 3, 2, or 1 to 1, 2, 3, 4, 5, 6, 7, 8, 9, 10, 15 or 20 and is preferably from 10:1 to 4:1. In certain aspects, the alkene is ethylene and the product includes ethylbenzene. In a further aspect, the alkene is propylene and the product stream includes cumene.

### II. EXAMPLES

The following examples as well as the figures are included to demonstrate certain aspects of the invention. It should be appreciated by those of skill in the art that the techniques disclosed in the examples or figures represent techniques discovered by the inventors to function well in the practice of the invention, and thus can be considered to constitute a mode for its practice. However, those of skill in the art should, in light of the present disclosure, appreciate that many changes can be made in the specific examples which are disclosed and still obtain a like or similar result without departing from the spirit and scope of the invention.

### Example 1

### Syntheses of a Hollow Fe-Silicalite-1 Based Catalyst

Synthesis of a hollow Fe-Silicalite-1 based catalyst are described. The characterization carried out on this material clearly showed that an iron particle was detectable within the hollow particles. No iron oxide particle were detected outside the hollow. An improvement of the catalyst synthesis includes metallic particle size and on the metal dispersion. This catalyst can be very selective for ethylbenzene or cumene synthesis.

A. Protocol

As an example, the protocol of the hollow zeolite with Fe oxide catalyst is described. A zeolite was treated with the corresponding template in the hydroxide form, tetrapropylammonium hydroxide (TPA(OH), SigmaMillipore) to obtain a MFI zeolite structure. The mixture was transferred into polytetrafluoroethylene lined autoclave and heated at 180° C. for 72 hours. Then the material was recovered by centrifugation (15 min at 10,000 rpm) and washed several times with water to remove the excess of template. After drying the material at 100° C. under air for 10 hours, the zeolite was calcined 6 hours at 540° C. (1° C./min) under air in order to clean the zeolite pores.

The metal complex incorporation was carried out by using wet impregnation method. Fe(III)Nitrate (0.637) was dissolved in methanol (2 mL) and then mixed with silicalite-1 (1.34 g). A special washing procedure was used to insure removing the metals from the surface of zeolite and not from inside the cavity. Since silicalite-1 is hydrophobic, water was selected to dissolve the metal on the external surface of zeolite because it cannot diffuse inside the cavities. The washing was carried out by adding water (8 to 12 mL) to the mixture, shaking, centrifuging at 100 rpm, and then removing the liquid phase. The final material was dried at 80° C. for 4 hours, and then calcined at 550° C. (1° C./min) for 6 hours under air.

B. Characterization

The synthesized catalyst were systematically characterized by using XRD diffraction, TEM analysis and N₂ adsorption desorption. Powder XRD patterns were recorded on an Empyrean from PANalytical using a nickel-filtered CuKα X-ray source, a convergence mirror and a PIXcelld detector. The scanning rate was 0.01° over the range between 5° and 80° 2θ. Imaging was performed using a Titan G2 80-300 kV transmission electron microscope operating at 300 kV (FEI Company) equipped with a 4 k×4k CCD camera, a GIF Tridiem (Gatan, Inc.) and an EDS detector (EDAX). N₂ adsorption/desorption isotherms were collected at 77K using Micromeretics ASAP 2010 apparatus. Before the measurement, approximately 100 mg of sample was degassed under vacuum (10 to 6 bar) at 350° C. for 10 hours.

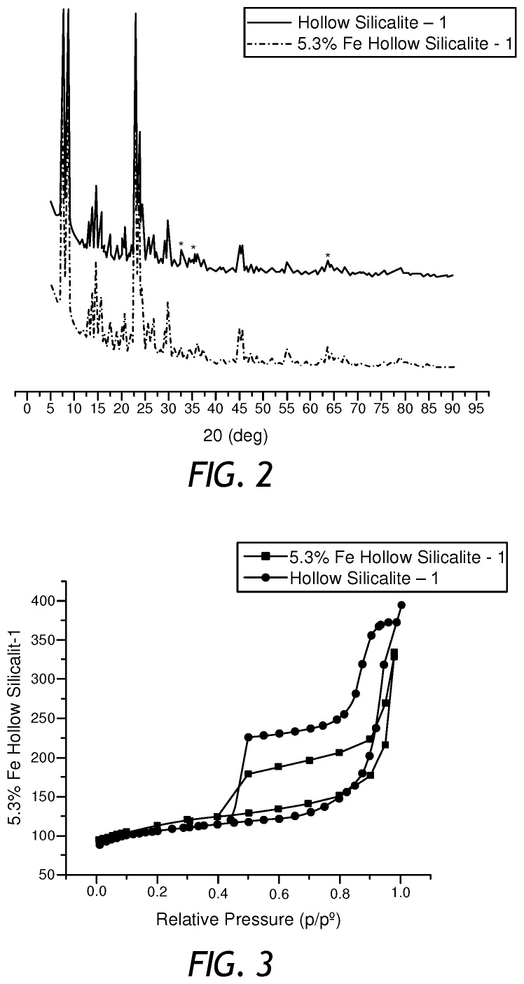

XRD analysis showed that the crystal structure was kept intact (FIG. 2) and zeolite crystals were not affected by hollow formation treatment and metal encapsulating. The peak assigned with the star were attributed to the Fe₂O₃. The other peaks are attributed to the silicalite-1 zeolite with MFI crystal structure.

FIG. 3 shows the N₂ Isotherm for 5.3% Fe hollow silicalite-1 and hollow silicalite-1 without metal. Of note, the hysteresis of the 5.3% Fe hollow silicalite-1 was smaller than the hollow silicalite-1 without metal. This was due to the metal oxide particle that filled the empty space and, consequently, decrease the pore volume and the hysteresis.

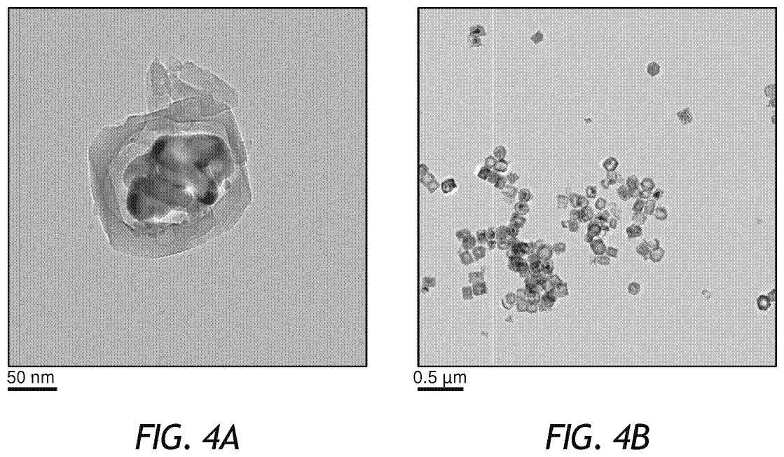

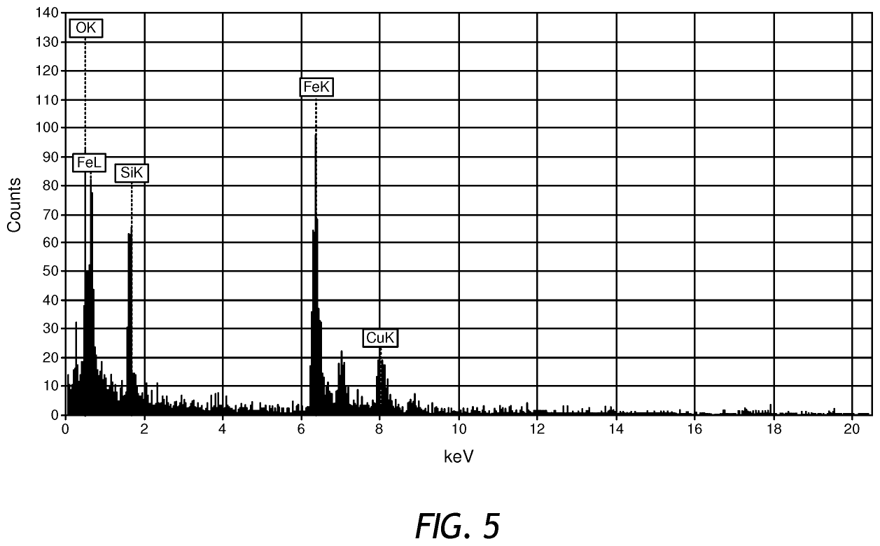

FIGS. 4A and 4B show TEM images of Fe incorporated within a hollow silicalite-1 and shows that iron oxide was successfully encapsulated within the cavities of silicalite-1. Also the washing used in the treatment was effective at removing metals from the external surface and not from the cavities as demonstrated in FIG. 4B. In addition, most of the silicalite-1 cavities encapsulate iron oxide, but still some cavities are empty and have no metal. This may be caused by the wet impregnation method used or the aggregation of the zeolite particle. This result leads to a non-uniform metal distribution within the zeolite. This non-uniformity can be avoided by a better controlled wet impregnation step. Also silicalite-1 could be aggregated before it was mixed with the metal salt solution, thus only the cavities in the outer surface of this aggregation were accessible to the metals. To overcome the aggregation effect the zeolite can be sonicated in a solvent such as methanol or ethanol before impregnation with the metal or actually performing the wet impregnation step during sonication.

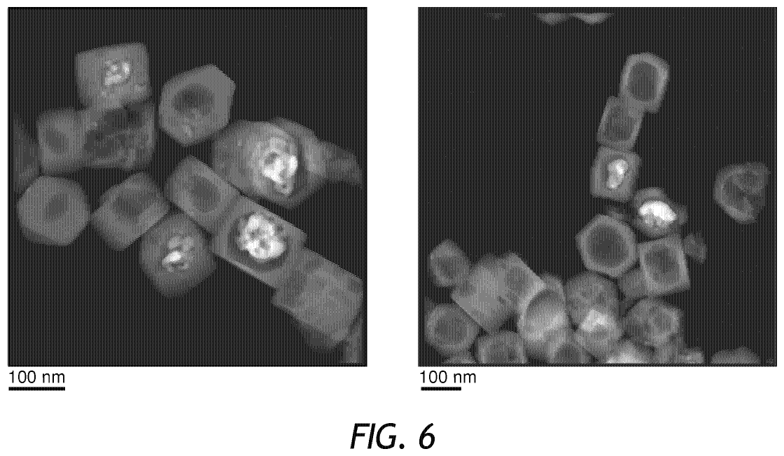

The catalyst was also characterized by dark field microscopy, see FIG. 6. The results are in good agreement with TEM, iron oxide incorporated inside hollow zeolite, no Fe particles on the surface and some cavities having no Fe oxide.

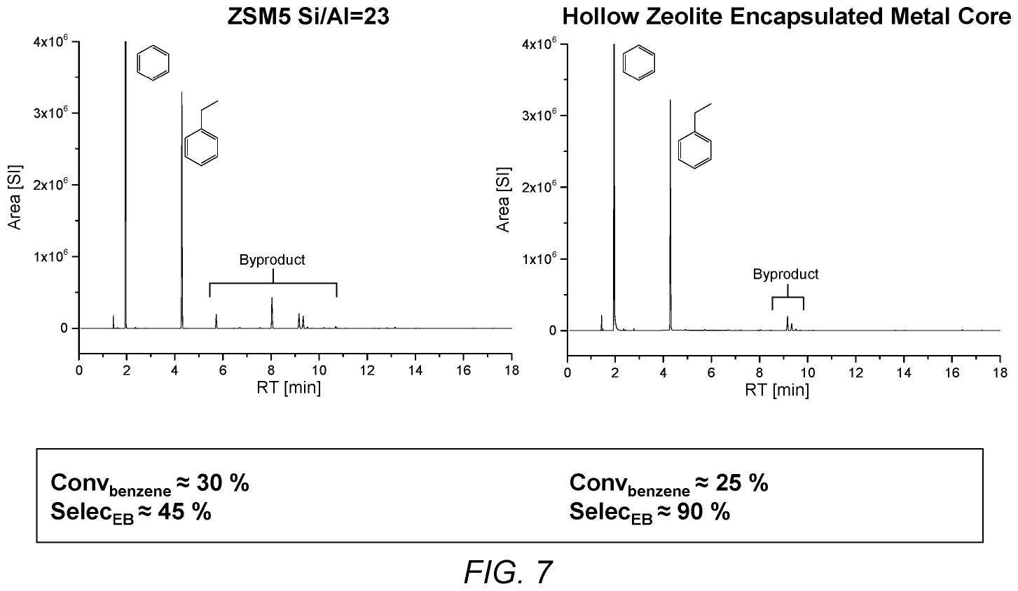

FIG. 7 shows results from a benzene alkylation reaction. Ethylbenzene was formed when an ethylene molecule reacts with a benzene ring in the presence of an acidic site to form ethylbenzene in a reaction known as alkylation (see Reaction 1 below). However, In the presence of extra ethylene molecules which can also react with ethylbenzene thus creating double or triple alkylation products.

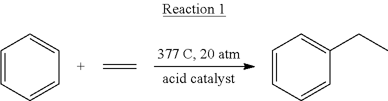

The methods of the invention described herein can achieve high yield and selective synthesis of ethylbenzene, which minimizes further alkylation steps. The catalysts (ZSM5 Si/Al=23 and a hollow zeolite encapsulated Fe particle), were tested using the following test parameters: type of reactor—Parr batch reactor with stirring; volume of reactor was 100 mL; volume of benzene was 10 mL (in excess to avoid polymerization of ethylene); pressure of ethylene at room temperature was about 10 bars (1 MPa); about 300 mg of catalyst; reaction temperature was 250° C.; reaction pressure was about 35-40 bar (3.5 to 4.0 MPa); and reaction time was 24 to 72 hours. After completion of the reaction, the pressure drop in the reactor gives an approximate conversion rate of ethylene and GC/MS analysis of the recovered liquid illustrates the selectivity for ethylbenzene. (See, FIG. 7). The ethylbenzene selectivity for the catalyst of the present invention was about 90% as compared to a 45% ethylbenzene selectivity for a ZSM-5 zeolite. The benzene conversion using the catalyst of the present invention was about 25%.

The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

1. A catalyst comprising a hollow zeolite encapsulated metal particle, where the metal particle is contained in the core of the hollow zeolite,
wherein the metal particle core comprises at least 97 wt. % of the metal present in the hollow zeolite encapsulated metal particle; and
wherein the metal particle precursor comprises iron, silver, gold, titanium, copper, zinc, cobalt, manganese, magnesium, nickel, palladium, iridium, ruthenium, aluminum, tungsten, bismuth, vanadium, indium, or combinations or alloys thereof.

2. The catalyst of claim 1, wherein the metal particle core comprises at least 98 wt. % of the metal present in the hollow zeolite encapsulated metal particle.

3. A method for producing a hollow zeolite catalyst according to claim 1 comprising:
(a) depositing a metal particle precursor in a hollow zeolite material by contacting the hollow zeolite material with a metal particle precursor that permeates or is transported into the hollow zeolite material and deposits the metal particle precursor in the hollow zeolite forming a hollow zeolite encapsulating a metal particle;
(b) removing metal particle precursor on the surface of the hollow zeolite while retaining the deposited metal particle precursor in the hollow portion of the hollow zeolite by contacting the hollow zeolite encapsulated metal particle with a non-permeating wash solution containing a solvent able to dissolve the metal complex but that does not substantially enter or permeate inside the hollow portion of the zeolite material forming a loaded hollow zeolite;
(c) drying the loaded hollow zeolite; and
(d) calcining the loaded hollow zeolite at 450 to 650° C. forming the hollow zeolite catalyst comprising a metal particle containing core wherein the metal particle core comprises at least 97 wt % of the metal present in the hollow zeolite encapsulated metal particle.

4. The method of claim 3, wherein steps (a) and (b) are repeated 2, 3, 4, 5, 6, 7, 8, 9, 10 or more times prior to the calcination step.

5. The method of claim 3, wherein the metal particle precursor comprises iron, silver, gold, titanium, copper, zinc, cobalt, manganese, magnesium, nickel, platinum, palladium, iridium, ruthenium, aluminum, tungsten, bismuth, vanadium, indium, or combinations or alloys thereof.

6. The method of claim 5, wherein the metal particle precursor is a metal oxide or metal salt.

7. The method of claim 6, wherein the metal particle precursor is a nitrate, chloride, sulfate, ammonium, acetate, or oxalate.

8. (canceled)

9. The method of claim 8, wherein the alcohol solution is an ethanol or methanol solution.

10. The method of claim 3, wherein the depositing step comprises wet impregnation, dry impregnation, vacuum impregnation, or ion exchange.

11. The method of claim 3, wherein the hollow zeolite is a type MFI, *BEA, MWW, or FAU zeolite.

12. The method of claim 3, wherein the hollow zeolite to metal particle precursor weight ratio in step (a) is 4:1 to 1:4.

13. The method of claim 12, wherein the hollow zeolite to metal particle precursor weight ratio in step (a) is 2:1 to 1:2.

14. The method of claim 3, further comprising forming a hollow zeolite by treating a zeolite with a corresponding template-hydroxide forming a first reaction mixture, and heating the first reaction mixture to a temperature of 150 to 200° C. for 24-120 hours forming a hollow zeolite material;
wherein the zeolite is a MFI type zeolite and the corresponding template-hydroxide is tetrapropylammonium hydroxide, a *BEA type zeolite and the corresponding template-hydroxide is tetraethylammonium hydroxide, a FAU type zeolite and the corresponding template-hydroxide is tetramethylammonium hydroxide, or a MWW type zeolite and the corresponding template-hydroxide is hexamethyleneimine hydroxide.

15. The method of claim 3, wherein the metal particle precursor is in an aqueous solution and the wash solution consists of water.

16. The method of claim 3, wherein the catalyst is substantially aluminum free such that aluminum is at levels that are not detectable using standard x-ray diffraction techniques.

17. The method of claim 3, wherein the catalyst has a silica to alumina molar ratio of 20:1 to ∞.

18. A method for alkylating benzene comprising contacting benzene with an alkene in the presence of the hollow zeolite catalyst of claim 1 at a temperature of 20 to 350° C.

19. The method of claim 18, wherein the alkene is ethylene and the product is ethylbenzene.

20. The method of claim 18, wherein the alkene is propylene and the product is cumene.
